(19) United States
(12) Patent Application Publication
(10) Pub. No.: US 2018/0231464 A1
(43) Pub. Date: Aug. 16, 2018

(54) ASSAY DEVICE

(71) Applicant: Molecular Vision Limited, York (GB)

(72) Inventors: Chris Hand, York (GB); Oliver Hofmann, Greater London (GB); Gihan Ryu, Surrey (GB); Miguel Ramon, Cambridgeshire (GB); Dene Baldwin, York (GB)

(73) Assignee: Molecular Vision Limited, York (GB)

(21) Appl. No.: 15/751,074

(22) PCT Filed: Aug. 19, 2016
(86) PCT No.: PCT/GB2016/052589

§ 371 Date: Feb. 7, 2018

(30) Foreign Application Priority Data
Aug. 19, 2015 (GB) 1514706.9

(51) Int. Cl. G01N 21/64; G01N 21/84; G01N 33/558; G01N 21/78

(52) U.S. Cl. CPC G01N 21/645; G01N 21/8483; G01N 2021/7786; G01N 33/558; G01N 21/78; G01N 21/6428

(57) ABSTRACT

An assay device for the quantitative determination of the concentration of at least one analyte in a liquid sample comprises a planar emitter (2), a planar detector (3), a lateral flow membrane (4) interposed between the emitter (2) and the detector (3), a conjugate pad (5) in fluid communication with a proximal end of the lateral flow membrane (4), the conjugate pad (5) comprising optically detectable tagging particles bound to a first assay component, a sample pad (6) in fluid communication with the conjugate pad (5) and arranged to receive the liquid sample, and a wicking pad (7) in fluid communication with a distal end of the lateral flow membrane (4). The lateral flow membrane (4) is formed from a light transmissive material and is capable of transporting fluid from the conjugate pad (5) to the wicking pad (7) by capillary action. The lateral flow membrane (4) comprises at least one test region (8, 12) comprising an immobilised second assay component for retaining the tagging particles in the test region (8, 12) in dependence on the binding between the analyte, the first assay component and the second assay component in order to generate a concentration of tagging particles in the test region (8, 12) that is indicative of the concentration of the analyte in the liquid sample. The emitter (2) comprises an emission layer (9, 16) of an organic electroluminescent material and the emission layer (9, 16) is aligned with the test region (8, 12) of the lateral flow membrane 4, whereby the emitter (2) is capable of illuminating the test region (8, 12). The detector (3) comprises an absorption layer (10, 15) of an organic photovoltaic material and the absorption layer (10, 15) is aligned with the test region (8, 12) of the lateral flow membrane 4, whereby the detector (3) is capable of detecting light from the test region (8, 12).

This invention relates to an assay device for the quantitative determination of the concentration of at least one analyte in a liquid sample. The liquid sample may be a biological sample, e.g. plasma, serum or urine. The sample may alternatively be a sample reduced to a liquid, such as a plant or tissue extract.

### BACKGROUND

Lateral flow devices (LFDs) have considerable use. One of the applications is in devices which analyse a liquid sample to determine the presence or absence of one or more target analytes which may be in the sample. In these devices, there is usually a threshold concentration which, when exceeded, results in an indication that a target analyte is present or absent.

Several techniques have been developed for producing a quantitative measurement of the concentration of the target analyte, for example using light receptors coupled with a light source. Within this field, there are two broad sub-classes. One of these uses the detection of the reflected emission from the light source. In this way, both the light source and the light detector are provided on the same side of the lateral flow membrane. An alternative technique positions the light source and the light detector on opposite sides of the lateral flow membrane, such that the light (or other electromagnetic radiation) must be transmitted through the membrane.

WO 2005/111579 is a transmission-based luminescent detection system.

The present invention, at least in its preferred embodiments aims to provide an alternative to devices of the prior art.

### BRIEF SUMMARY OF THE DISCLOSURE

In accordance with the present invention there is provided an optical detection unit for an assay device for the quantitative determination of the concentration of at least one analyte in a liquid sample. The detection unit comprises a planar emitter, a planar detector, and a lateral flow membrane interposed between the emitter and the detector and comprising a plurality of test regions. Each test region comprises an immobilised assay component. The planar emitter comprises an emission layer of an organic electroluminescent material and the emission layer is aligned with the plurality of test regions of the lateral flow membrane, whereby the emitter is capable of illuminating each of the test regions. The planar detector comprises an absorption layer of an organic photovoltaic material and the absorption layer is aligned with the plurality of test regions of the lateral flow membrane, whereby the detector is capable of detecting light from each of the test regions. The emission layer comprises 15 or more emitter pixels and the detection layer comprises 15 or more detector pixels. Each said test region is interposed between one said emitter pixel and one said detector pixel whereby to form an optical pathway from the emitter pixel to the respective detector pixel through at least a portion of the respective test region.

Thus, the optical detection unit can be used in an assay device to perform at least 15 different tests on a liquid sample. In some cases, the tests may be the same tests or different tests or a mixture of the two. The immobilised assay component may be the same in each test region or different immobilised assay components may be provided in respective test regions.

The planar emitter and/or the planar detector may further comprise surface conductors for the addressing of the emitter pixels and the detector pixels. The surface conductors may be configured for the separate addressing of each emitter pixel and each detector pixel.

Where the emission layer or detection layer comprises 15 pixels, the pixels may be arranged in three rows of five pixels. Alternatively, the pixels may be arranged in five rows of three pixels. The emission layer may comprise at least 21 pixels. The detection layer may comprise at least 21 pixels. Where the emission layer or detection layer comprises 21 pixels, the pixels may be arranged in three rows of seven pixels. Alternatively, the pixels may be arranged in seven rows of three pixels.

In some embodiments, each row may be offset by half the pixel spacing relative to adjacent rows. Thus, the pixels may be arranged to maximise the distance between adjacent pixels for a given area of the planar emitter or planar detector. Thus, optical cross-talk between pixels (where an emission from one emitter pixel has an effect on the signal measured by an adjacent detector in the region of a different emitter pixel) is minimised.

It will be appreciated that other configurations of pixels are possible where the total number of pixels is 15, 21, or any other number of pixels greater than 15.

In accordance with an aspect of the present invention, there is provided an assay device comprising the optical detection unit.

In accordance with an aspect of the present invention, there is provided a method for measuring the light absorption of each test region of the optical detection unit. The method comprises addressing in a predetermined sequence the emitter pixel and detector pixel forming the optical pathway through each test region.

The predetermined sequence may address only a subset of the emitter pixels at any one time. The predetermined sequence may address only a subset of the detector pixels at any one time.

The predetermined sequence may address a plurality of detector pixels and/or a plurality of emitter pixels having interposed therebetween test regions comprising the same immobilised assay component simultaneously.

The predetermined sequence may address the emitter pixels and/or the detector pixels one at a time.

The predetermined sequence may address a plurality of detector pixels and/or emitter pixels each separated by at least a threshold distance, whereby to minimise cross-talk between the pixels.

The predetermined sequence may address all detector pixels and emitter pixels.

There is disclosed herein an assay device for the quantitative determination of the concentration of at least one analyte in a liquid sample. The device comprises a planar emitter, a planar detector, a lateral flow membrane interposed between the emitter and the detector, a conjugate pad in fluid communication with a proximal end of the lateral flow membrane, the conjugate pad comprising optically detectable tagging particles bound to a first assay component, and a wicking pad in fluid communication with a distal end of the lateral flow membrane. The lateral flow membrane is formed from a light transmissive material and is capable of transporting fluid from the conjugate pad to the wicking pad by capillary action. The lateral flow membrane comprises at least one test region comprising an immobilised second assay component for retaining the tagging particles in the test region in dependence on the binding between the analyte, the first assay component and the second assay component in order to generate a concentration of tagging particles in the test region that is indicative of the concentration of the analyte in the liquid sample. The emitter comprises an emission layer of an organic electroluminescent material and the emission layer is aligned with the test region of the lateral flow membrane, whereby the emitter is capable of illuminating the test region. The detector comprises an absorption layer of an organic photovoltaic material and the absorption layer is aligned with the test region of the lateral flow membrane, whereby the detector is capable of detecting light from the test region.

Thus, in accordance with the invention, the assay device provides a relatively simple construction that is capable of determining the result of an assay by optical measurement of the test region. Embodiments of the invention are capable of accurately determining the concentration of an analyte in a sample. However, it is not necessary in every embodiment of the invention for the device to determine the exact concentration of the analyte. For example, in some embodiments only a qualitative indication of the analyte concentration may be determined. Typically, however, embodiments of the invention provide more than a simple yes/no indication of the presence of the analyte. The device improves upon the prior art by the ability to provide a quantitative indication of the concentration in a device that can be configured for single-use.

At least one of the test regions may be in the shape of a substantially rectangular line. Alternatively, at least one of the test regions may be a circle, square or dot. It will be appreciated that the test regions may be supplied in any conceivable shape fitting within the boundary of the lateral flow membrane.

In an embodiment of the invention, the tagging particles absorb light at a wavelength emitted by the emitter, and the detector is arranged to detect light from the emitter passing through the lateral flow membrane, whereby the attenuation of the light intensity detected by the detector due to absorption by the immobilised tagging particles is indicative of the concentration of the analyte in the liquid sample. For example, the tagging particles may be gold nanoparticles which appear red when concentrated and may be illuminated by green light from the emitter. As a further example, the tagging particles may be blue polystyrene particles and may be illuminated by red light from the emitter. The light from the emitter may be in the visible spectrum, but could also be in the ultraviolet or infra red wavelength ranges.

In an embodiment of the invention, the tagging particles fluoresce under illumination at a wavelength emitted by the emitter, and the detector is arranged to detect such fluorescence through the lateral flow membrane, whereby the light intensity detected by the detector due to fluorescence of the immobilised tagging particles is indicative of the concentration of the analyte in the liquid sample. For example, the tagging particles may be fluorescein or fluorescein isothiocyanate (FITC) particles illuminated with blue light.

The light transmissive material may become light transmissive when wetted by the liquid sample. The light transmissive material may be nitrocellulose. This material has been found to be particularly suitable. When dry, nitrocellulose is substantially opaque. However, when wet, the nitrocellulose may become light transmissive. In this way, the nitrocellulose is particularly suitable for use in head-on detection geometry, since light can be transmitted through the lateral flow membrane when wet. The lateral flow membrane may have a thickness of less than 200 microns, preferably less than 150 microns, more preferably less than 100 microns.

The spacing between the facing surfaces of the emission layer and the absorption layer may be less than 1.5 mm, preferably less than 1 mm, more preferably less than 0.5 mm. Close spacing of the emission layer and the absorption layer maximises the amount of captured light and therefore maximises the signal to noise ratio of the device.

The spacing between the facing surfaces of the emission layer and the lateral flow membrane may be less than 1 mm, preferably less than 0.5 mm, more preferably less than 0.2 mm. Close spacing of the emission layer and the lateral flow membrane maximises the intensity of the emitted light at the membrane and therefore maximises the signal to noise ratio of the device.

The spacing between the facing surfaces of the absorption layer and the lateral flow membrane may be less than 1 mm, preferably less than 0.5 mm, more preferably less than 0.2 mm. Close spacing of the absorption layer and the lateral flow membrane maximises the intensity of the incident light at the detector and therefore maximises the signal to noise ratio of the device.

The emitter may comprise an electrode layer interposed between the emission layer and the lateral flow membrane. The electrode layer of the emitter may comprise indium tin oxide. Typically, the emitter may be made up of a plurality of layers, including anode and cathode layers. The emitter may comprise a barrier layer interposed between the electrode layer and the lateral flow membrane. The barrier layer may be provided by a substrate on which the emitter is formed. The barrier layer can protect the emission layer during construction of the device. The barrier layer may be the only layer between the electrode layer and the lateral flow membrane. In embodiments of the invention there is no air gap between the emitter and the lateral flow membrane. This minimises the distance the light must travel from the emission layer to the lateral flow membrane.

The detector may comprise an electrode layer interposed between the absorption layer and the lateral flow membrane. The electrode layer of the detector may comprise indium tin oxide. Typically, the detector may be made up of a plurality of layers, including anode and cathode layers. The detector may comprise a barrier layer interposed between the electrode layer and the lateral flow membrane. The barrier layer may be provided by a substrate on which the detector is formed. The barrier layer can protect the absorption layer during construction of the device. The barrier layer may be the only layer between the electrode layer and the lateral flow membrane. In embodiments of the invention there is no air gap between the detector and the lateral flow membrane. This minimises the distance the light must travel from the lateral flow membrane to the absorption layer.

The emitter and/or the detector may be formed by deposition, in particular printing, of layers on a substrate. In one embodiment, the emitter and the detector are each provided on separate substrates. The substrate may be flexible, for example PET, or may be rigid, for example glass. In a particularly advantageous embodiment the emitter and the detector are formed on a common substrate. The substrate may be folded about the lateral flow membrane. By depositing both the emitter and the detector on the same substrate correct relative alignment of the emitter and the detector can be ensured.

This in itself is believed to be novel and thus, viewed from a further aspect the invention provides an electro-optical device comprising an emitter comprising an organic electroluminescent material and a detector comprising an organic photovoltaic material, wherein the electroluminescent material and the photovoltaic material are deposited on a common substrate.

Typically, the emission layer comprises an organic electroluminescent material, such as polymers including poly(p-phenylene vinylene) or polyfluorene, or small molecules including organometallic chelates, fluorescent or phosphorescent dies, and conjugated dendrimers. The organometallic chelate may be Alq₃. The absorption layer typically comprises an organic photovoltaic material, such as the small molecules PCBM₆₀ or PCBM₇₀, or polymers such as polythiophenes. The absorption layer may comprise a blend of organic photovoltaic polymers such as polythiophenes and organic photovoltaic small molecules such as PCBM₆₀ or PCBM₇₀. The polythiophene may be Poly(3-hexylthiophene) (P3HT).

The assay device may further comprise a sample pad in fluid communication with the conjugate pad and arranged to receive the liquid sample. The conjugate pad may perform the role of a sample pad, where no distinct sample pad is provided.

In an embodiment of the invention, the lateral flow membrane comprises a plurality of discrete test regions and the emission layer comprises a plurality of discrete emission regions each aligned with a respective test region. Similarly, the lateral flow membrane may comprise a plurality of discrete test regions and the absorption layer may comprise a plurality of discrete absorption regions each aligned with a respective test region. In this way each test region may be provided with a respective emission region and/or a respective detection region. By providing discrete emission or absorption regions, respective test regions can be analysed independently and the risk of cross talk is minimised.

The lateral flow membrane may comprise a control region. The control region may be positioned between the test region(s) and the distal end of the lateral flow membrane, the control region may comprise an immobilised control component for retaining tagging particles in the control region and the emission layer and/or the absorption layer may comprise a discrete emission/absorption region aligned with the control region.

The first assay component may comprise a molecule which binds the analyte to the tagging particles and the second assay component may comprise a receptor for the analyte. This combination of components is useful in a sandwich assay.

The first assay component may comprise the analyte or an analogue thereof and the second assay component may comprise a receptor for the analyte. This combination of components is useful in a competitive assay. Alternatively, the first assay component comprises a receptor for the analyte and the second assay component comprises the analyte or an analogue thereof. The assay may be an immunoassay. The receptor may be an antibody which binds to the analyte or an analogue thereof.

The lateral flow membrane is provided on a transparent substrate. The substrate may provide mechanical stability to the lateral flow membrane.

The assay device may comprise a controller arranged to receive detection signals from the detector and to process the detection signals whereby to generate data indicative of the concentration of the analyte in the sample. The controller may be provided as part of the assay device, for example within the same housing. The controller may also be arranged to control the emission of light from the emitter. The device may comprise a battery for powering the detector and the emitter. The device may be disposable.

The device may comprise an electrical interface for connection to an external reader, wherein the electrical interface is configured to connect the detector and the emitter to the external reader. In this way, the device can be provided as a disposable cartridge.

The assay device may comprise at least a second lateral flow membrane arranged in parallel with the first lateral flow membrane between the emitter and the detector.

Thus, in accordance with an embodiment of the invention, a second lateral flow membrane allows multiple assay tests to be performed in parallel. In some embodiments, the multiple assay tests may be testing for the same analyte in the same way. Alternatively, the multiple assay tests may be testing for different analytes. Performing assay tests in parallel prevents the mechanism of one assay test interfering with the mechanism of a second assay test.

The second lateral flow membrane may be provided on the same sheet as the first lateral flow membrane. The second lateral flow membrane may be joined to the first lateral flow membrane. Alternatively, the second lateral flow membrane may be provided separately to the first lateral flow membrane.

The wicking pad may be in fluid communication with a distal end of the first lateral flow membrane and a distal end of the second lateral flow membrane. Thus, the first lateral flow membrane and the second lateral flow membrane both connect to the same wicking pad.

The conjugate pad may be in fluid communication with a proximal end of the first lateral flow membrane and a proximal end of the second lateral flow membrane. Thus, the first lateral flow membrane and the second lateral flow membrane both connect to the same conjugate pad.

The conjugate pad may comprise optically detectable tagging particles bound to a third assay component.

The optically detectable tagging particles bound to the third assay component may be optically different to the optically detectable tagging particles bound to the first assay component. Thus, the different colours of the optically detectable tagging particles allow two tests to be run in close proximity without the spectrum-matched light required to test the result of one test interfering with the spectrum-matched detector required to test the result of the second, neighbouring test.

The assay device may comprise a second conjugate pad in fluid communication with a proximal end of the second lateral flow membrane.

The second conjugate pad may comprise optically detectable tagging particles bound to a third assay component. The second conjugate pad may comprise optically detectable tagging particles bound to the first assay component.

The optically detectable tagging particles in the second conjugate pad may be optically different to the said optically detectable tagging particles in the first conjugate pad. Thus, the different colours of the optically detectable tagging particles allow two tests to be run in close proximity without the spectrum-matched light required to test the result of one test interfering with the spectrum-matched detector required to test the result of the second, neighbouring test.

In some embodiments, the second lateral flow membrane may comprise at least a second test region comprising an immobilised fourth assay component for retaining the tagging particles in the second test region in dependence on the binding between the analyte, the third assay component and the fourth assay component.

In some embodiments, the second lateral flow membrane may comprise at least a second test region comprising the immobilised first assay component for retaining the tagging particles in the second test region in dependence on the binding between the analyte, the first assay component and the second assay component.

The (first) lateral flow membrane may comprise at least a second test region comprising an immobilised fourth assay component for retaining the tagging particles in the second test region in dependence on the binding between the analyte, a (said) third assay component and the fourth assay component.

The emission layer may comprise a plurality of emitter pixels and a first emitter pixel may be aligned with the (first) test region of the first lateral flow membrane and a second emitter pixel may be aligned with the second test region.

The absorption layer may comprise a plurality of detector pixels and a first detector pixel may be aligned with the (first) test region of the first lateral flow membrane and a second detector pixel may be aligned with the second test region. The second test region may be provided on the first lateral flow membrane or the second lateral flow membrane.

The first emitter pixel and the second emitter pixel may be mutually spaced in the direction from the distal end to the proximal end of the lateral flow membrane.

The first detector pixel and the second detector pixel may be mutually spaced in the direction from the distal end to the proximal end of the lateral flow membrane.

The first detector pixel may be aligned with the first emitter pixel and the second detector pixel is aligned with the second emitter pixel.

Thus, the mutual spacing of the emitter and/or detector pixels minimises the amount of light from the first emitter pixel detectable in the second detector pixel or vice versa.

The pixels may be defined as discrete regions of the emission layer or the absorption layer. Alternatively, the emission layer or the absorption layer may be masked to define the pixels. However, this is not preferred.

### BRIEF DESCRIPTION OF THE DRAWINGS

Embodiments of the invention are further described hereinafter with reference to the accompanying drawings, in which:

FIG. 1A is an illustration of an assay device according to an embodiment of the present invention;

FIG. 1B is an illustration of a further view of an assay device according to the embodiment of FIG. 1A;

FIG. 2 is an illustration of an assay device according to a further embodiment of the present invention;

FIG. 3 is an illustration of a component of an embodiment of an assay device according to the present invention;

FIG. 4 is an illustration of a 1-row pixel pattern of an embodiment of an assay device according to the present invention;

FIG. 5 is an illustration of a 2-row pixel pattern of an embodiment of an assay device according to the present invention;

FIG. 6 is an illustration of a 3-row pixel pattern of an embodiment of an assay device according to the present invention;

FIG. 7 is an illustration of a 4-row pixel pattern of an embodiment of an assay device according to the present invention;

FIGS. 8a and 8b show the dose response curves of Kappa and Lambda FLC assays according to Example 1;

FIG. 9 shows the dose response curves of an opiate assay according to Example 2; and

FIGS. 10 and 11 are illustrations of a 5-row and 7-row pixel pattern of an embodiment of an optical detection unit according to an embodiment of the present invention.

### DETAILED DESCRIPTION

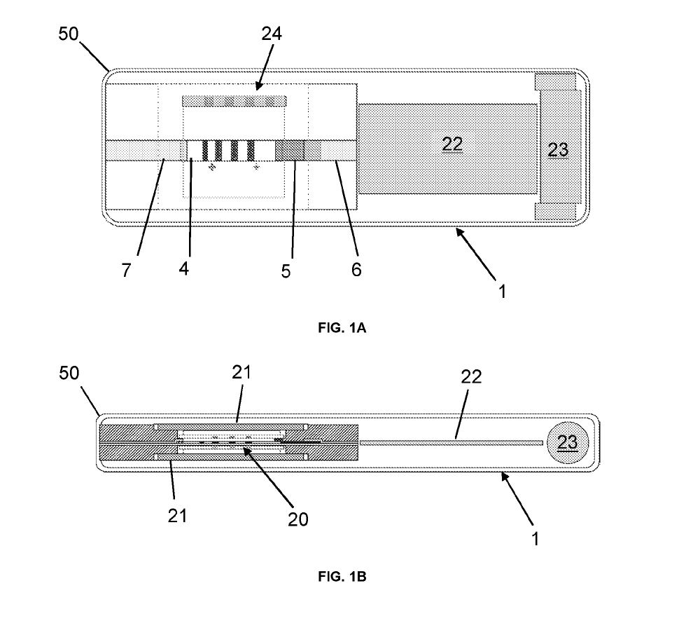

As shown in FIG. 1A and FIG. 1B, according to one embodiment of the present invention, there is provided an assay device 1 contained in a thin, substantially cuboidal housing 50. FIG. 1B provides a side-on illustration of the schematic diagram for the same device as illustrated in FIG. 1A. One end of the housing contains a testing module 20 provided in the plane of the length and width of the housing 50. The opposite end of the housing 50 accommodates a cylindrical battery 23 flat against the wall of the housing 50. Between the testing module 20 and the battery 23 is a printed circuit board 22 which extends from the battery into the length of the housing in the same plane as the testing module 20. Electronics in the testing module 20 are connected to the printed circuit board 22 via an electrical interface 24. The testing module 20 contains a sample pad 6, in fluid communication with a conjugate pad 5. The present conjugate pad 5 contains particle tags which are capable of binding to an assay component. A lateral flow membrane 4 is connected between the conjugate pad 5 and a wicking pad 7. A support structure 21 secures the testing module 20 in the housing 50.

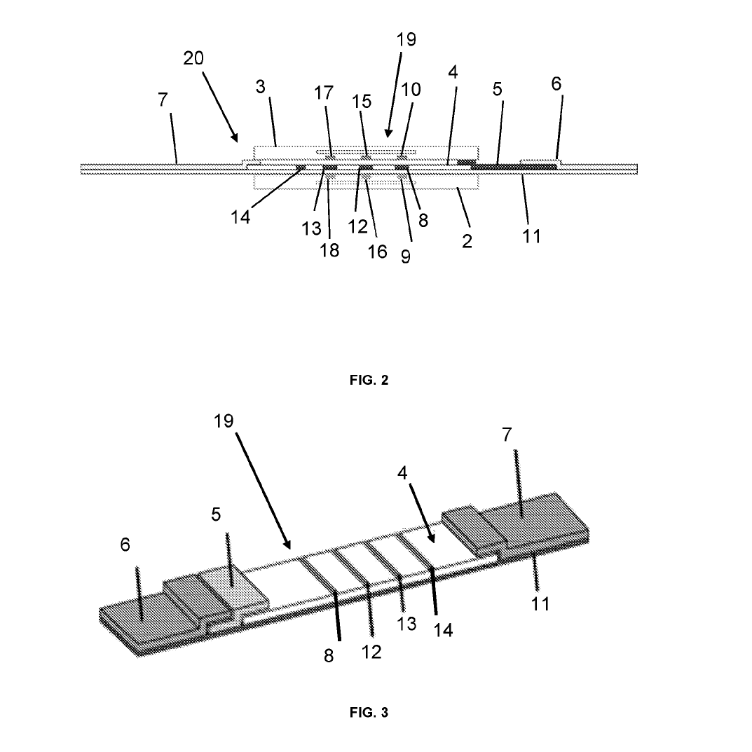

FIG. 2 illustrates a testing module 20 according to an embodiment of the present invention. When a sample is deposited on the sample pad 6, a reservoir of excess sample is formed. The excess sample migrates to the conjugate pad 5. This migration is first caused by the conjugate pad 5, then the wicking action of the lateral flow membrane 4 and then additionally the wicking pad 7. The lateral flow membrane 4 is formed from nitrocellulose. The conjugate pad 5 contains analyte tags. The analyte tags bind to the corresponding available analyte. Capillary action causes the liquid sample, containing any tagged analytes, to flow down the lateral flow membrane 4 from the conjugate pad 5 into the testing area 19 towards the wicking pad 7. Before the sample reaches the wicking pad 7, it encounters a reaction line 8 containing fixed receptors for the analyte. When the tagged analyte reaches this point, the receptors bind to the analyte, holding the analyte and the tags in place. The presence of the coloured analyte tag will cause the reaction line 8 to change colour as the concentration of the tags increases. In the presently described example, the concentration of the coloured tags is a direct indicator of the concentration of analyte at the reaction line which provides an indication of the concentration of the analyte in the liquid sample.

The above is an example of a sandwich assay technique. A competitive assay is also possible in which the intensity of the response from the reaction line 12 (usually a colour) is inversely proportional to the amount of analyte present in the sample. In one example of this technique, the conjugate pad 5 additionally contains a pre-tagged second analyte or analyte analogue. The analyte from the sample passes unchanged through the conjugate pad 5, and will bind to the receptors on a further reaction line 12, occupying receptor sites to which the pre-tagged analytes or analyte analogues would otherwise bind. The less analyte there is in the sample, the more pre-tagged analyte or analyte analogue is able to bind to the receptors, resulting in a stronger colouring of the line. In a further example of this technique, the conjugate pad 5 could also or instead contain a tagged receptor. In this case fixed analyte or analyte analogue is immobilised on a reaction line. The more analyte present in the sample, the more of the tagged receptor that will bind to the analyte from the sample, and so not be available to bind to the fixed analyte or analyte analogue. The competitive assay technique may be used to qualitatively test for the absence of a particular analyte, though is not a purely binary test, and a very small amount of analyte in the sample is still likely to result in binding of the pre-tagged molecule (be that analyte, analyte analogue or receptor) at the position of the line. The competitive assay technique may instead be used to quantitatively indicate the concentration of a particular analyte in the liquid sample.

There is also a further line 13 of control receptors on the lateral flow membrane 4 which react with the tagged component itself. The control line 13 contains immobilised receptors which bind to the tagged component. The control line 13 should become coloured whenever the test is carried out, regardless of whether the sample contains any analyte. This helps confirm the test is performing correctly. In the presently described example, the reaction line 8 only changes colour when the analyte is present in the sample. In embodiments with multiple assays, there may be multiple control lines. In this way, the control lines can be used to determine whether each test to be performed by the lateral flow device has been performed. The control line 13 in the current example is provided downstream of the earlier reaction lines. By providing the control line 13 downstream of the reaction lines, the analyte tag must flow through the other reaction lines before they can bind to the control line indicating that a test has been carried out.

In the present case, the lateral flow membrane 4 is approximately 100 μm thick and the reaction lines 8, 12 and control line 13 are each 1.0 mm×5.0 mm with a 2.0 mm gap between them. The lateral flow membrane is formed from nitrocellulose. The sample pad 6, conjugate pad 5, lateral flow membrane 4 and wicking pad 7 are provided on a transparent substrate 11.

A reference line 14 is provided on the lateral flow membrane 4 and is used for alignment during construction of the testing area 19. The reference line 14 is typically thinner than the reaction lines 8, 12 or control line 13. The reference line in the current example is 0.5 mm×5.0 mm with a 1.5 mm gap between the control line 13.

Whilst the examples disclose analysing the presence, absence, or concentration of a range of analytes in the sample, it is possible to perform this analysis with fewer or more analyte tests. A range of different tags and receptor lines can be used to determine the presence, absence, or concentration of multiple different analytes. The presence of some analytes may be tested in combination with the absence of different, or the same, analytes. Tests for example assays are given in Table 1 below. In each case, the purpose of the test is given, along with the first assay component, second assay component, the analyte of interest, and which type of assay (sandwich or competitive). All assays can be performed using analyte or antibodies to the analyte labelled with any type of labelling particle. Example labelling particles include gold nano-particles, coloured latex particles, or fluorescent labels. As can be readily identified from the table in row N, assays for other analytes can be constructed using analyte antigens as the first component and antibodies to the analyte as the second component where the assay type is sandwich. Where the assay type is competitive (row M), the antibodies to the analyte would be the first component, and the analyte antigen would be the second component.

TABLE 1

| | | | Label Binder | Immobilised Line | | Assay Type |
|---|---|---|---|---|---|---|
| | | | (first | (second | | (Sandwich/ |
| | Test for: | Label | component) | component) | Analyte | Competitive) |
| A | Myeloma | All | Antibodies to free | Kappa FLC | Kappa FLC | Competitive |
| | | | kappa light chains | antigen | | |
| | | | (k-FLC) | | | |
| B | Myeloma | All | Antibodies to free | Lambda FLC | Lambda FLC | Competitive |
| | | | lambda light | antigen | | |
| | | | chains (l-FLC) | | | |
| C | Myeloma | All | Antibodies to free | Antibodies to free | Kappa FLC | Sandwich |
| | | | kappa light chains | kappa light chains | | |
| | | | (k-FLC) | (k-FLC) | | |
| D | Myeloma | All | Antibodies to free | Antibodies to free | Lambda FLC | Sandwich |
| | | | lambda light | lambda light | | |
| | | | chains (l-FLC) | chains (l-FLC) | | |
| E | Opiates | All | Antibodies to | Opiates antigen | Opiates | Competitive |
| | | | Opiates | | | |
| F | Amphetamines | All | Antibodies to | Amphetamines | Amphetamines | Competitive |
| | | | Amphetamines | antigen | | |
| G | Benzodiazepines | All | Antibodies to | Benzodiazepines | Benzodiazepines | Competitive |
| | | | Benzodiazepines | antigen | | |
| H | Cannabis | All | Antibodies to | Cannabinoid | Cannabis | Competitive |
| | | | Cannabinoids | derivative antigen | | |
| I | Cocaine | All | Antibodies to | Cocainoids | Cocaine | Competitive |
| | | | Cocainoids | antigen | | |
| J | Methamphetamine | All | Antibodies to | Methamphetamine | Methamphetamine | Competitive |
| | | | Methamphetamine | antigen | | |
| K | Methadone | All | Antibodies to | Methadone | Methadone | Competitive |
| | | | Methadone | antigen | | |
| L | Phencyclidine | All | Antibodies to | Phencyclidine | Phencyclidine | Competitive |
| | (PCP) | | Phencyclidine | (PCP) antigen | (PCP) | |
| | | | (PCP) | | | |
| M | Others | All | Antibodies to | Others antigen | Others | Competitive |
| | | | Others | | | |
| N | Others | All | Antibodies to | Antibodies to | Others | Sandwich |
| | | | Others | Others | | |
| O | Troponin I | All | Antibodies to | Antibodies to | Troponin I | Sandwich |
| | | | Troponin I | Troponin I | | |
| P | Myoglobin | All | Antibodies to | Antibodies to | Myoglobin | Sandwich |
| | | | Myoglobin | Myoglobin | | |
| Q | CKMB | All | Antibodies to | Antibodies to | CKMB | Sandwich |
| | | | CKMB | CKMB | | |
| R | Cortisol in saliva, | All | Antibodies to | Cortisol antigen | Cortisol | Competitive |
| | serum or urine | | Cortisol | | | |

Whilst common household assay tests, such as some pregnancy tests, have an apparently binary result and require a user to manually interpret the results, the present device uses an Organic Light Emitting Diode (OLED) and opposed Organic Photo Diode (OPD) to measure the light absorption as a result of the analyte test. Whilst the presently described embodiment uses the absorption of light by a substance to indicate the concentration of an analyte in a test sample, embodiments can equally be envisaged where the tag on the analyte is luminescent and emits light itself, either as a result of fluorescence, phosphorescence, or as a result of a chemical or electrochemical reaction.

The assays for Myeloma are described in rows labelled A-D in Table 1. To test for myeloma, the ratio of Kappa FLC concentration to Lambda FLC concentration is determined.

The OLED illuminates the sample with light having known characteristics (intensity, wavelength, etc). When light is received by the OPD, a current is generated. By measuring this current, the light absorbed by the immobilised labels at the reaction line, 8, 12 and surrounding membrane can be determined. This gives an indication of the concentration of tagged analyte present in the sample.

The OLED is a layered structure sitting on a plastic substrate (PET). The OLED is formed from a layer of patterned ITO (indium tin oxide, which is conductive and transparent), a layer of hole injection material, a layer of active material, and a cathode. It is possible to maximize the forward emission of the device by tuning the thicknesses of the ITO and more importantly the active material and cathode. With such modifications in the stack geometry the amount of light being emitted perpendicular to the device can be maximised. This will mean that a larger proportion of light emitted by the OLED passes through the membrane, and impinges onto the OPD. Conventional inorganic LEDs with epoxy protection have a lambertian emission, and therefore waste a significant amount of light.

In the present example, the OLED 2 contains emission regions 9, 16, 18, provided opposite the organic photovoltaic cell (OPD) 3, containing detection regions 10, 15, 17.

The emission light colour of all three regions in the present example is blue, as they are formed from a layer of the same material. Similarly, in the present example, the material of the OPD regions 10, 15, 17 is optimised to detect blue light.

The OLED emission regions 9, 16, 18 and OPD detection regions 10, 15, 17 are sized to sit within the footprint of the reaction lines 8, 13, 14 containing bound receptors set up to catch and bind the tagged analyte (be that pre-tagged or otherwise). In the present case, this results in pixels 0.9 mm×4.9 mm. This maximises the proportion of the light emission from the OLED that is capable of interacting with the tagged analyte and the surrounding lateral flow membrane 4. Another factor which improves the proportion of the emitted light that can interact with the membrane and tagged analyte is the proximity of both the OLED and the OPD to the lateral flow membrane 4. In the present example, only the barrier material is interposed between the OLED/OPD and the membrane, with a thickness of approximately 100 μm.

The circuit board 22 and battery 23 included within the housing 50 for the assay device 1 control and power the OLED and OPD. The circuit board 22 also includes a microprocessor suitable for performing basic analysis in order to calculate a quantitative value representative of the amount of the analyte(s) present in the sample and/or ratios thereof.

For an example OPD the following structure can be used. The first layer (closest to the membrane) is a pre-patterned indium-tin-oxide (ITO) glass substrate. The glass substrate provides a barrier layer for the OPD. On top of the ITO layer is provided a 50 nm thick layer of Baytron P grade poly(styrenesulphonate)-doped poly(3,4-ethylenedioxythiophene) (PEDOT:PSS) and 10 nm thick Poly(methyl methacrylate) (PMMA) film interlayer is provided thereon. The active layer is 165 nm thick regioregular poly(3-hexylthiophene):1-(3-Methoxycarbonylpropyl)-1-phenyl-[6.6]061 (P3HT:PCBM) with an upper electrode for the device of 100 nm-thick aluminium.

This is only one example of an OPD suitable for use in embodiments of the present invention. The skilled person will be aware of methods of manufacturing such OPDs and other materials from which suitable OPDs may be manufactured.

The skilled person is aware of several methods and material combinations from which to fabricate OLEDs suitable for the present invention. In one particular OLED type, the structure is a plastic substrate (PET), a layer of patterned ITO, a layer of hole injection material, a layer of active material, and a cathode. In particular, the spectrum output of the OLED can be selected by the correct choice of the organic polymer or other small molecule.

The spectrum of emission of the OLED must be matched to the absorbance of the relevant light quencher (the coloured tags used to label the compound of interest). In an absorbance regime, gold nanoparticles can be used. In this case, a green illumination source should be used. Alternatively, blue polystyrene labels can be used. In this case, a red illumination source should be used. In a fluorescence regime, fluorescein/FITC based labels can be used. In this case, a blue illumination source should be used.

Furthermore, the forward emission of the OLED can be maximised by tuning the thicknesses of the ITO, active material and cathode. Maximising the forward emission ensures that a maximised amount of the light emitted by the OLED is emitted perpendicular to the active surface of the device. In this way, there is a maximised proportion of the light emitted by the OLED which passes through the light quenchers and onto the OPD. This increases both the sensitivity and accuracy of these devices.

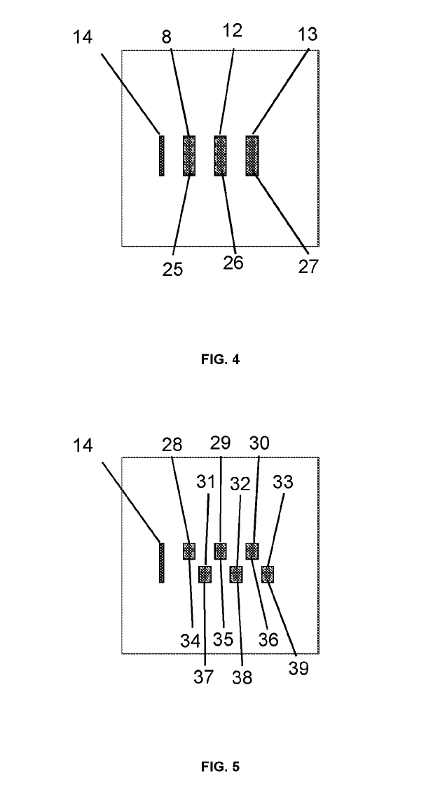

FIG. 4 illustrates a 1-row pixel pattern of an embodiment of an assay device according to the present invention. The reference line 14, reaction lines 8 and 12, and control line 13 are provided on the lateral flow membrane. The OLED and OPD production processes allow pixels of any size and positioning to be created to overlay the reaction and control lines. In FIG. 4, the pixel outlines 25, 26, and 27 shown as dashed lines represent the outline of the OPD sensitive regions and OLED pixels. These pixels are centred on the reaction lines 8, 12 (or control line 13). The pixel outlines 25, 26, and 27 are also smaller than the reaction lines 8, 12 (or control line 13). In this way, the light which enters the OPD from the OLED without passing through the reaction line (i.e. passing through a part of the lateral flow membrane not forming part of the reaction line or control line) is minimised and/or substantially eliminated. In some embodiments, the pixel outlines may have substantially the same extent as the reaction lines. The reaction lines 8, 12 may be correspond to assays for the same analyte. In this way, the accuracy of any resulting indications of the analyte concentration in the liquid sample can be maximised by multiple assays of the same sample.

FIG. 5 illustrates a 2-row pixel pattern of an embodiment of an assay device according to the present invention. In this embodiment, there are two parallel lateral flow membranes. As described previously, the reference line 14 is used to align the reaction regions 28, 29, 30, 31, 32, 33 with the OPD and OLED outlines 34, 35, 36, 37, 38, 39 respectively. By diagonally offsetting the matched reaction regions (lines) from each other, the light bleed between two neighbouring reaction regions, is minimised. In this way, for example, the amount of light from the OPD/OLED outline 37 detectable by the OPD on the OPD/OLED outline 34, 35 is minimised. This allows a particularly compact arrangement of assays in a single assay device. In some embodiments, each parallel lateral flow membrane can contain a single reaction region, with each lateral flow membrane testing for a different analyte. In other embodiments, each parallel lateral flow membrane can contain a single or multiple reaction regions, with each lateral flow membrane testing for the same one or group of analytes. This allows the accuracy of the resulting indications of the analyte concentrations in the liquid sample to be improved. In yet other embodiments, multiple testing regions on a plurality of parallel lateral flow membranes can be used to test for the same analyte in different ways. In this way, one lateral flow membrane may test for a given analyte using a sandwich assay technique, whilst another lateral flow membrane may test for the same given analyte using a competitive assay technique.

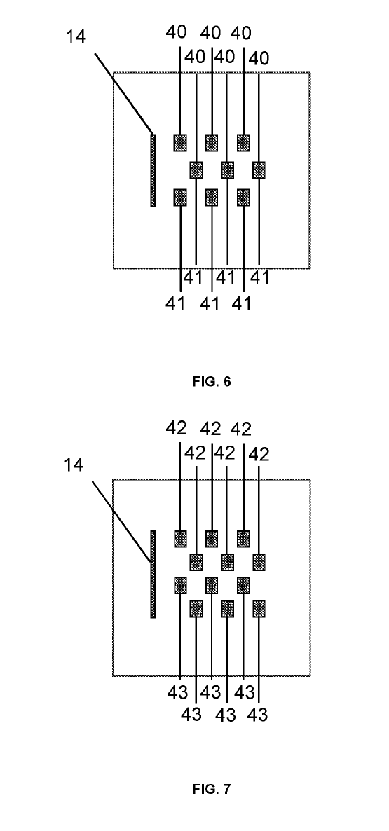

FIGS. 6 and 7 illustrate respectively a 3-row and 4-row pixel pattern of an embodiment of an assay device according to the present invention. The reaction regions 40, 42 provided on the lateral flow membrane are arranged to minimise light from the OLED having outline 41, 43 bleeding into the outline of any neighbouring OPD having outline 41, 43. As before, the reference line 14 is provided for alignment purposes.

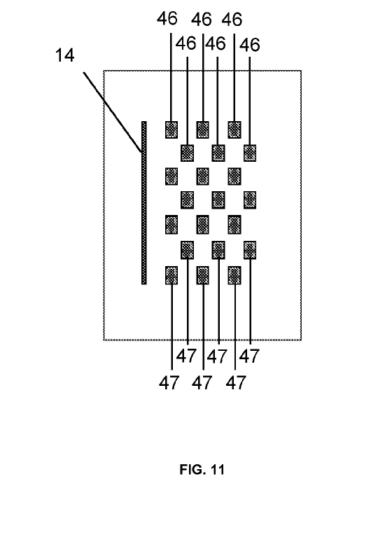

FIGS. 10 and 11 illustrate respectively a 5-row and 7-row pixel pattern of an embodiment of an optical detection unit according to the present invention. The reaction regions 44, 46 provided on the lateral flow membrane are arranged to minimise light from the OLED having outline 45, 47 bleeding into the outline of any neighbouring OPD having outline 45, 47. As before, the reference line 14 is provided for alignment purposes. In particular, FIG. 10 shows an embodiment of a pixel pattern comprising a total of 15 emitter pixels and 15 detector pixels. Although the presently illustrated embodiments show lines of 3 pixels, it will be appreciated that alternative configurations may be provided having, for the embodiment shown in FIG. 10, lines of 5 pixels, but in 3 rows, which would also have a total of 15 pixels. For the embodiment shown in FIG. 11, having 21 pixels, it will be appreciated that an alternative embodiment may involve lines of 7 pixels, but in 3 rows. Alternatively, different pixel patterns may be provided as necessary. In some embodiments, more than 21 pixels may be provided by the pixel pattern.

The OLEDs and OPDs also comprise surface conductors for the separate addressing of each light emitting diode and each photodiode. The OLEDs and OPDs are addressed in a predetermined sequence to minimise the effect of an OPD detecting emission originating from an OLED other than that paired with the OLED and provided the other side of the associated test region on the lateral flow membrane. In one embodiment, the OLEDs and OPDs are addressed one at a time in sequence. In another embodiment, the OLEDs and OPDs are addressed in such a way that when a first OLED/OPD pair is addressed (and so activated), none of the OLEDs or OPDs adjacent to the first OLED/OPD pair are simultaneously addressed. In another embodiment, the OLED/OPD pairs may be addressed such that only OLED/OPD pairs corresponding to tests for the same analyte are activated simultaneously.

Whilst in the embodiments shown, the reaction lines and/or reaction regions are intended to extend to each side of each lateral flow membrane, as seen specifically in reaction line 12 from FIG. 3, the invention extends to alternative embodiments where the reaction lines and/or reaction regions do not extend to each side of each lateral flow membrane. For example, the reaction regions may be centred in the middle of the lateral flow membrane. Alternatively, two distinct regions may be provided side-by-side on a lateral flow membrane. There may be a space on the lateral flow membrane between the two reaction regions. In some embodiments, the two reaction regions are provided in contact with each other. In some embodiments, two or more regions may be spaced or offset both in the proximal-distal direction, and in the width direction of the lateral flow membrane. The reaction regions may be provided on distinct lateral flow membranes which may be provided, for example, side-by-side.

Whilst embodiments of the present invention have been described using direct tagging, indirect tagging is also possible. In embodiments where a first antibody binds to the analyte, the tagging particle may be bound to a further antibody, which is configured to bind to the first antibody. In this way the same labelled antibody can be used for several different analytes.

Whilst the embodiments shown use a conjugate pad, it will be appreciated that the sample may be pre-treated with the analyte tags. This may ensure better mixing and binding between the analyte and analyte tags, particularly where there are very low concentrations of analyte. In this case, the conjugate pad is not required, and the pre-treated sample may be deposited on the sample pad or the lateral flow membrane directly. In some embodiments where the presence or concentration of multiple analytes is to be tested, the sample may be pre-treated for only some of the analytes of interest. In this case, a conjugate pad is still required.

Whilst the embodiments shown are for quantitative measurements, it will be appreciated that the invention is equally applicable to qualitative or semi-quantitative assay devices, where only a presence or absence indication of one or more analytes of interest is required. In semi-quantitative assay devices, only a discretised reading of, for example, a plurality concentration levels is required. The concentration levels need not be regularly spaced over the range of concentration to be measured.

An advantage of the present invention in embodiments using fabricated OPDs and OLEDs compared to prior art devices using silicon-based inorganic detectors or GaAs and/or InGaAs and/or SbGaInAs-based inorganic emitters is the ability to provide multiple assays (quantitative or otherwise) without a corresponding increase in material costs. In the inorganic emitters and detectors of the prior art, multiple reaction regions require multiple emitters and detectors, which each have a unit cost. In embodiments of the present invention, OPDs and OLED are fabricated from a single piece, regardless of the number of pixels the emitter or detector requires, and so there is only a minimal increase in cost for the provision of an additional reaction region.

### Example 1

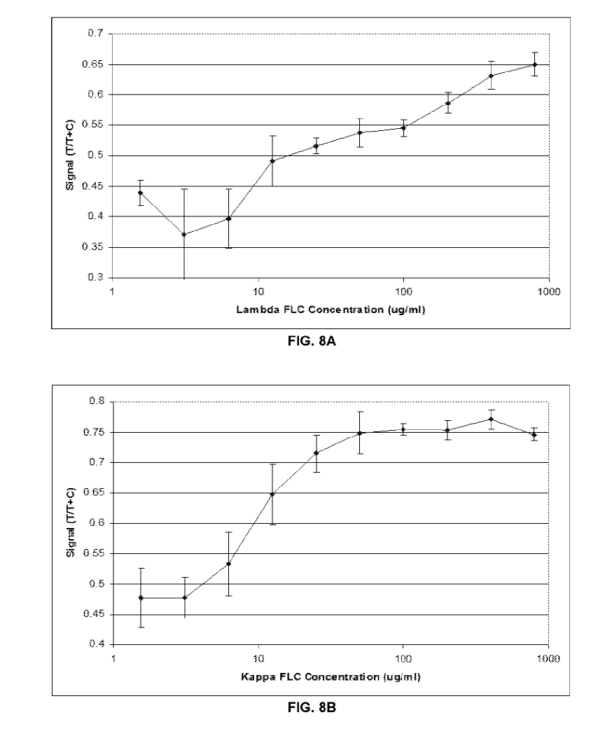

An organic light emitting diode (OLED) has three pixels in the manner of the embodiment of FIG. 4 and emits green light with a wavelength of 520 nm and an organic photo diode (OPD) has the same pattern as the OLED. The lateral flow membrane comprises one control region and two test regions. The first assay is Kappa FLC antigen and the second assay is Lambda FLC antigen. When an amount of a sample containing Kappa and Lambda FLC antigen flows along the membrane, tagged antibodies combine with Kappa and Lambda FLC antigens in the sample or on the membrane. More antigens in the sample generate less colour and more light is transmitted through the membrane so that a larger signal is detected by the OPD. FIG. 8 shows the dose response curves of the Kappa and Lambda FLC assays.

### Example 2

An organic light emitting diode (OLED) has a configuration as shown in FIG. 5 but only two of three pixels are operated in each row. The emitting wavelength is 520 nm.

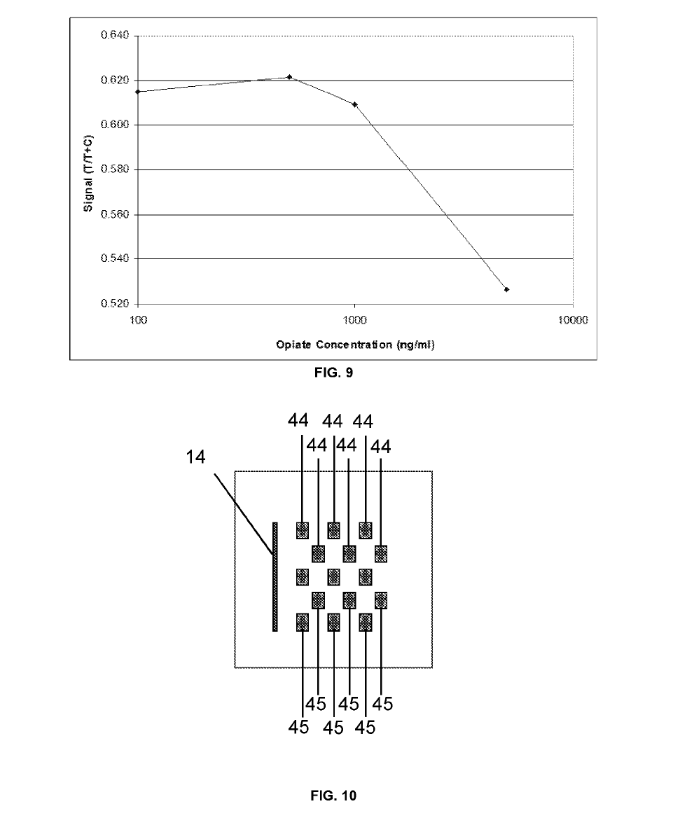

The organic photo diode (OPD) has the same pattern as the OLED. The lateral flow membrane comprises one control region and one test region of opiates antibody. Two identical lateral flow membrane stripes are aligned in parallel with two rows of OLED and OPD pairs to improve the accuracy by running samples twice simultaneously. When a sample including a certain amount of opiates antigen flows along the membrane, the antigen combines with tagging material (gold beads) and binds with opiates antibody on the membrane. More antigens in the sample generate darker colour and less light transmits through the membrane so that weaker signal is detected by the OPD. FIG. 9 is a dose response curve for the opiates assay.

In summary, an assay device for the quantitative determination of the concentration of at least one analyte in a liquid sample comprises a planar emitter 2, a planar detector 3, a lateral flow membrane 4 interposed between the emitter 2 and the detector 3, a conjugate pad 5 in fluid communication with a proximal end of the lateral flow membrane 4, the conjugate pad 5 comprising optically detectable tagging particles bound to a first assay component, a sample pad 6 in fluid communication with the conjugate pad 5 and arranged to receive the liquid sample, and a wicking pad 7 in fluid communication with a distal end of the lateral flow membrane 4. The lateral flow membrane 4 is formed from a light transmissive material and is capable of transporting fluid from the conjugate pad 5 to the wicking pad 7 by capillary action. The lateral flow membrane 4 comprises at least one test region 8,12 comprising an immobilised second assay component for retaining the tagging particles in the test region 8,12 in dependence on the binding between the analyte, the first assay component and the second assay component in order to generate a concentration of tagging particles in the test region 8,12 that is indicative of the concentration of the analyte in the liquid sample. The emitter 2 comprises an emission layer 9,16 of an organic electroluminescent material and the emission layer 9,16 is aligned with the test region 8,12 of the lateral flow membrane 4, whereby the emitter 2 is capable of illuminating the test region 8,12. The detector 3 comprises an absorption layer 10,15 of an organic photovoltaic material and the absorption layer 10,15 is aligned with the test region 8,12 of the lateral flow membrane 4, whereby the detector 3 is capable of detecting light from the test region 8,12. Embodiments of the present invention allow for the fabrication of fully disposable quantitative multi-zone diagnostic devices ideally suited for home testing.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An optical detection unit for an assay device for the quantitative determination of the concentration of at least one analyte in a liquid sample, the detection unit comprising:
a planar emitter;
a planar detector; and
a lateral flow membrane interposed between the emitter and the detector and comprising a plurality of test regions, each test region comprising an immobilised assay component,
wherein the planar emitter comprises an emission layer of an organic electroluminescent material and the emission layer is aligned with the plurality of test regions of the lateral flow membrane, whereby the emitter is capable of illuminating each of the test regions, and
wherein the planar detector comprises an absorption layer of an organic photovoltaic material and the absorption layer is aligned with the plurality of test regions of the lateral flow membrane, whereby the detector is capable of detecting light from each of the test regions,
wherein the emission layer comprises 15 or more emitter pixels and wherein
the detection layer comprises 15 or more detector pixels, and
wherein each said test region is interposed between one said emitter pixel and once said detector pixel whereby to form an optical pathway from the emitter pixel to the respective detector pixel through at least a portion of the respective test region.

2. An optical detection unit as claimed in claim 1, wherein the emission layer comprises at least 21 pixels and wherein the detection layer comprises at least 21 pixels.

3. An optical detection unit as claimed in claim 1, wherein the planar emitter and planar detector each further comprise surface conductors for the separate addressing of each emitter pixel and each detector pixel.

4. An assay device comprising the optical detection unit of claim 1.

5. A method for measuring the light absorption of each test region of an optical detection unit as claimed in claim 3, the method comprising:
addressing in a predetermined sequence the emitter pixel and detector pixel forming the optical pathway through each test region.

6. A method as claimed in claim 5, wherein the predetermined sequence addresses only a subset of the emitter pixels and/or detector pixels at any one time.

7. A method as claimed in claim 5, wherein the predetermined sequence addresses a plurality of detector pixels and/or a plurality of emitter pixels having interposed therebetween test regions comprising the same immobilised assay component simultaneously.

8. A method as claimed in claim 5, wherein the predetermined sequence addresses the emitter pixels and/or the detector pixels one at a time.

9. A method as claimed in any of claim 5, wherein the predetermined sequence addresses a plurality of detector pixels and/or emitter pixels each separated by at least a threshold distance, whereby to minimise cross-talk between the pixels.

10. A method as claimed in claim 5, wherein the predetermined sequence addresses all detector pixels and emitter pixels.
